(19) United States
(12) Patent Application Publication
(10) Pub. No.: US 2026/0232294 A1
(43) Pub. Date: Aug. 13, 2026

(54) INFORMATION PROCESSING APPARATUS, INFORMATION PROCESSING SYSTEM, AND INFORMATION PROCESSING PROGRAM

(71) Applicant: NIHON KOHDEN CORPORATION, Tokyo (JP)

(72) Inventors: Hiraku TABATA, Saitama (JP); Mitsuhiro OURA, Saitama (JP); Sou KUMAGAI, Saitama (JP)

(73) Assignee: NIHON KOHDEN CORPORATION, Tokyo (JP)

(21) Appl. No.: 19/152,753

(22) PCT Filed: Jan. 31, 2024
(86) PCT No.: PCT/JP2024/003035

§ 371 Date: Jul. 31, 2025

(30) Foreign Application Priority Data
Feb. 9, 2023 (JP) 2023-018398

(51) Int. Cl. A61B 8/00; A61B 5/021; A61B 5/1455; A61B 5/318; A61B 8/02; A61B 8/06

(52) U.S. Cl. CPC A61B 8/4416; A61B 5/021; A61B 5/14551; A61B 5/318; A61B 8/02; A61B 8/06; A61B 8/465; A61B 8/488

(57) ABSTRACT

An information processing apparatus (2) includes an acquisition unit (2012) configured to acquire sensor information from each of a plurality of sensors including an ultrasonic sensor (3, 3A, 3B) coming into contact with or in proximity to a subject, and a derivation unit (2015) configured to derive a predetermined parameter indicating a body state of the subject based on the acquired sensor information.

### FIELD

The presently disclosed subject matter relates to an information processing apparatus, an information processing system including the information processing apparatus, and an information processing program.

### BACKGROUND

In recent years, diagnosis carried out using an ultrasonic sensor that includes a plurality of ultrasonic elements has become important (for example, Patent Literature 1). For example, ultrasonic images of blood vessels, bones, organs, or the like of subjects are produced using sensor information acquired from an ultrasonic sensor.

### CITATION LIST

### Patent Literature

Patent Literature 1: US2020/0337680A

### SUMMARY

### Technical Problem

It is desirable to ascertain body states of subjects (patients) diversely using sensor information acquired from such an ultrasonic sensor.

Accordingly, an object of the presently disclosed subject matter is to provide an information processing apparatus, an information processing system, and an information processing program capable of ascertaining a body state of a subject (patient) diversely.

### Solution to Problem

The problem of the presently disclosed subject matter is solved by the following means.

According to a first aspect of the presently disclosed subject matter, an information processing apparatus includes: an acquisition unit configured to acquire sensor information from each of a plurality of sensors including an ultrasonic sensor coming into contact with or in proximity to a subject; and a derivation unit configured to derive a predetermined parameter indicating a body state of the subject based on the acquired sensor information.

### Advantageous Effects of Invention

In the information processing apparatus according to the presently disclosed subject matter, a predetermined parameter indicating a body state of a subject is derived based on sensor information acquired from each of a plurality of sensors including an ultrasonic sensor. Accordingly, it is possible to ascertain a body state of a subject diversely.

### BRIEF DESCRIPTION OF DRAWINGS

FIG. 1 illustrates an example of a configuration of an information processing system according to a first embodiment of the presently disclosed subject matter.

FIG. 2A is a top view illustrating an ultrasonic sensor illustrated in FIG. 1.

FIG. 2B is a side view illustrating an ultrasonic sensor illustrated in FIG. 1.

FIG. 2C is a perspective view illustrating an ultrasonic sensor illustrated in FIG. 1.

FIG. 3 is a block diagram illustrating an example of a configuration of a control unit illustrated in FIG. 1.

FIG. 4 is a block diagram illustrating an example of a function of a CPU illustrated in FIG. 3.

FIG. 5 illustrates examples of parameters displayed on a display illustrated in FIG. 1.

FIG. 6 is a flowchart illustrating an example of a process of an information processing apparatus illustrated in FIG. 1.

FIG. 7 is a flowchart of a sub-routine of step S105 illustrated in FIG. 6.

FIG. 8 illustrates an example of a relation between an ultrasonic sensor illustrated in FIG. 2 and a blood vessel of a subject.

FIG. 9 is a diagram in which an XZ plan view illustrated in FIG. 8 is enlarged.

FIG. 10 illustrates parameters derived by the information processing system according to Modification Example 1.

FIG. 11 illustrates an example of a configuration of an information processing system according to a second embodiment of the presently disclosed subject matter.

FIG. 12 is a block diagram illustrating an example of a function of a control unit illustrated in FIG. 11.

FIG. 13 illustrates examples of parameters displayed on a display illustrated in FIG. 11.

### DESCRIPTION OF EMBODIMENTS

Hereinafter, an ultrasonic diagram system (ultrasonic processing system) according to an embodiment of the presently disclosed subject matter will be described in detail with reference to the drawings. In the drawings, the same reference numerals denote the same members. In the drawings, dimension ratios are exaggerated to facilitate description and are different from actual ratios in some cases.

### First Embodiment

### (Configuration of Information Processing System)

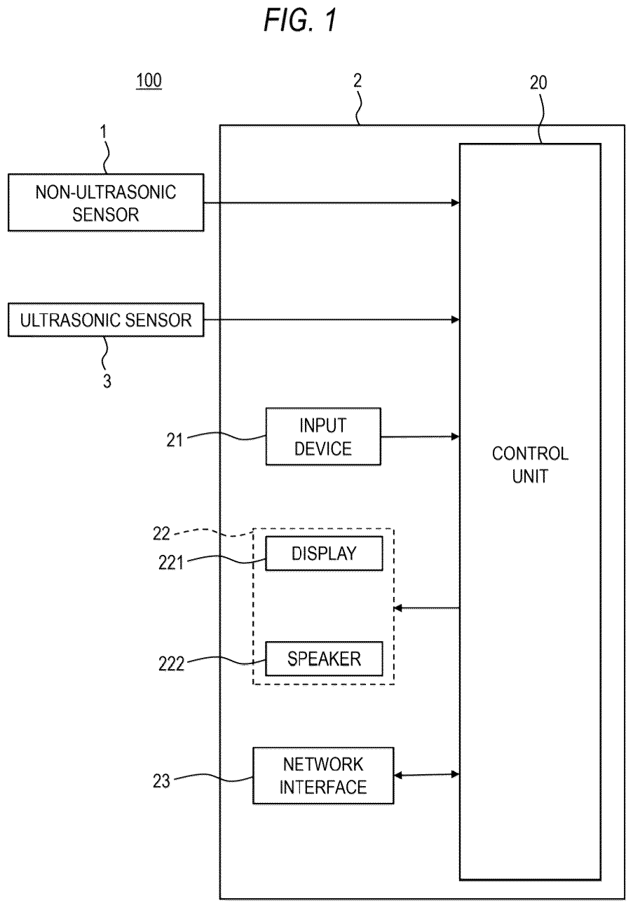

FIG. 1 illustrates an example of an overall hardware configuration of an information processing system 100 according to a first embodiment. The information processing system 100 includes a non-ultrasonic sensor 1, an information processing apparatus 2, and an ultrasonic sensor 3. The non-ultrasonic sensor 1 and the ultrasonic sensor 3 can be connected to the information processing apparatus 2. The non-ultrasonic sensor 1 and the ultrasonic sensor 3 come into contact with a subject or are disposed in proximity to the subject to detect a body state of the subject. Here, the proximity refers to a state in which the non-ultrasonic sensor 1 and the ultrasonic sensor 3 are disposed within a range in which a body state of the subject can be detected.

The information processing system 100 derives predetermined parameters indicating a body state of a subject (patient) using sensor information acquired from each of the non-ultrasonic sensor 1 and the ultrasonic sensor 3 by the information processing apparatus 2. Accordingly, a user of the information processing system 100 (for example, a medical practitioner such as a doctor or a nurse) can perform appropriate curing or treatment for the subject according to the derived predetermined parameters.

The non-ultrasonic sensor 1 is a sensor that detects physical information other than an ultrasonic wave and measures a body state of the subject. For example, the non-ultrasonic sensor 1 measures a body state of the subject using pressure, electricity, light, or the like. The non-ultrasonic sensor 1 includes, for example, a cuff or catheter and measures a blood pressure of the subject. The cuff is mounted so that an air bag is wound on an upper arm of the subject. The non-ultrasonic sensor 1 may include a pressurization pump and an exhaust valve along with the cuff. For example, the catheter is inserted into a blood vessel of the subject. The sensor information acquired from the non-ultrasonic sensor 1 includes, for example, information regarding a blood pressure of the subject.

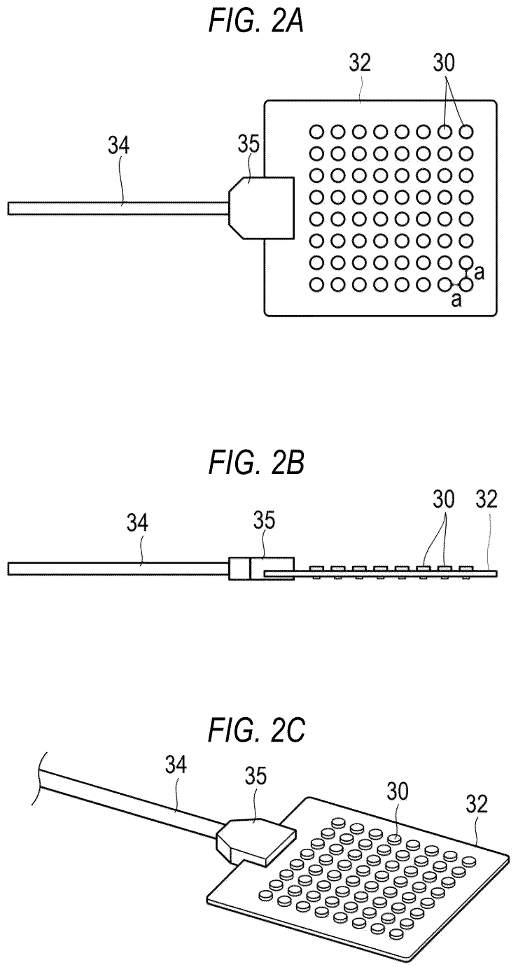

FIGS. 2A to 2C illustrate an example of a configuration of the ultrasonic sensor 3. FIG. 2A is a top view of the ultrasonic sensor 3, FIG. 2B is a side view, and FIG. 2C is a perspective view. The ultrasonic sensor 3 includes, for example, a plurality of ultrasonic elements 30, a sheet-shaped member 32 that has flexibility, and a connection member 35, and is connected to the information processing apparatus 2 via a cable 34. For example, the sheet-shaped member 32 is mounted on a body surface of the subject and an ultrasonic image of the subject is generated. For example, the sheet-shaped member 32 is mounted on a neck region of the subject.

The sheet-shaped member 32 has flexibility and is deformed along a shape of an attached portion when the sheet-shaped member 32 is attached to the body surface of the subject. The sheet-shaped member 32 has, for example, a rectangular planar shape (FIG. 2A). The sheet-shaped member 32 is formed of, for example, a resin material such as polyimide or silicon.

For example, the plurality of ultrasonic elements 30 are buried in a matrix form in the sheet-shaped member 32. The ultrasonic elements 30 may be disposed on the sheet-shaped member 32. In FIGS. 2A to 2C, 64 ultrasonic elements 30 disposed in 8 rows in the row direction and 8 columns in the column direction are illustrated, but the number and disposition of the ultrasonic elements 30 are not limited thereto. For example, 96 ultrasonic elements may be disposed in 3 rows in the row direction and 32 columns in the column direction.

For example, the plurality of ultrasonic elements 30 are disposed at uniform intervals on the sheet-shaped member 32 and are disposed in the row direction and the column direction at intervals a. The intervals of the adjacent ultrasonic elements 30 may be known, may be disposed at different intervals in the row direction and the column direction, or may be disposed at intervals different according to a position of the sheet-shaped member 32.

Each of the plurality of ultrasonic elements 30 includes, for example, a piezoelectric substance and an electrode. The ultrasonic element 30 transmits an ultrasonic wave in response to a transmission instruction sent from the information processing apparatus 2 via the cable 34. When the ultrasonic element 30 receives an ultrasonic wave reflected from a blood vessel, a bone, an organ, or the like (hereinafter referred to as a reflected wave) in the body of the subject, a signal is sent from each ultrasonic element 30 to the information processing apparatus 2 via the cable 34.

The connection member 35 connects the cable 34 and each of the plurality of ultrasonic elements 30 and is configured with, for example, a printed substrate board or the like.

The information processing apparatus 2 includes, for example, a patient monitor, a defibrillator or a respirator (FIG. 1). The information processing apparatus 2 may be a computer such as a server or a PC. The information processing apparatus 2 includes, for example, a control unit 20, an input device 21, an output device 22, and a network interface 23.

The input device 21 is configured to receive an input operation of a user operating the information processing apparatus 2 and generate an input signal corresponding to the input operation. The input device 21 can include, for example, a touch panel superimposed on a display 221 of the output device 22 to be described below, an operation button mounted on the casing of the information processing apparatus 2, a mouse, or a keyboard. The input signal generated by the input device 21 is transmitted to the control unit 20. The control unit 20 performs a predetermined process according to the input signal.

The output device 22 outputs information regarding a predetermined parameter derived by the control unit 20. The output device 22 can include, for example, the display 221 and a speaker 222. The display 221 can be, for example, a liquid crystal display or an organic EL display mounted on the casing of the information processing apparatus 2. The display 221 may be a display device such as a transmissive or non-transmissive head-mount display mounted on the head of the user.

The speaker 222 is mounted on the casing of the information processing apparatus 2 and outputs an alarm sound for the user. Information regarding the predetermined parameter may be output as a sound. The output device 22 includes a light-emitting unit including a light emitting diode (LED) and can be configured to output an alert by light of the LED or the like.

The output device 22 is not limited to the display 221 and the speaker 222. For example, a printer that prints and outputs information regarding the predetermined parameter can also be included.

The network interface 23 connects the control unit 20 to a communication network. Specifically, the network interface 23 includes a processing circuit for various interfaces for communicating with an external apparatus such as a server via the communication network and conforms with a communication standard for communication via the communication network. The communication network is a local area network (LAN), a wide area network (WAN), the Internet, or the like.

The control unit 20 derives the predetermined parameter using sensor information acquired from each of the non-ultrasonic sensor 1 and the ultrasonic sensor 3. The control unit 20 may be software and hardware that mainly control the information processing apparatus 2 or the control unit 20 may be an independent device. For example, the control unit 20 may be a dedicated medical device that derives the predetermined parameter or may be a personal computer, a smartphone, a tablet terminal, or the like in which an information processing program for deriving the predetermined parameter is installed. The control unit 20 may be a wearable device mounted on the body (for example, an arm, the head, or the like) of the user.

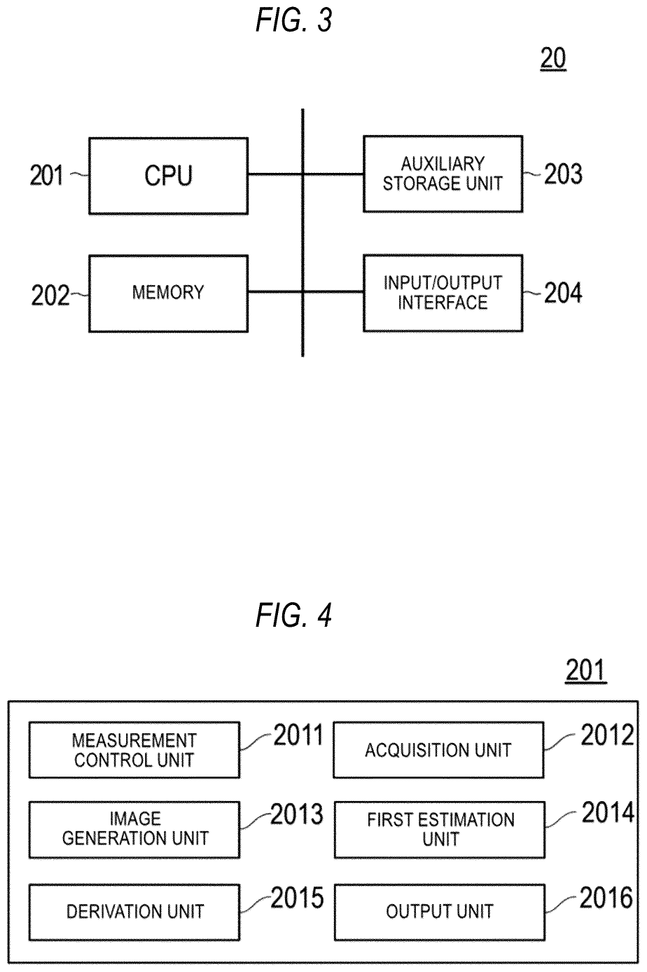

FIG. 3 is a block diagram illustrating an example of an overall hardware configuration of the control unit 20. The control unit 20 can include, for example, a central processing unit (CPU) 201, a memory 202, an auxiliary memory 203, and an input/output interface 204. For example, the memory 202 can include a read only memory (ROM) and a random access memory (RAM), The ROM stores various programs, parameters, and the like necessary to derive the predetermined parameter. The RAM can include a work area in which various programs or the like executed by the CPU 201 are stored. The CPU 201 is configured to load designated programs on the RAM among various programs stored in the ROM or the auxiliary memory 203 and execute various processes in cooperation with the RAM. The CPU 201 executes an information processing program and the control unit 20 controls each unit of the information processing system 100 so that various functions are implemented. The details of functions implemented by the CPU 201 will be described below.

The auxiliary memory 203 can include, for example, a storage device (storage) such as a hard disk drive (HDD), a solid state drive (SSD), or a USB flash memory. The auxiliary memory 203 is configured to store the information processing program or various types of data. The auxiliary memory 203 stores data related to a blood pressure, an ultrasonic image, and the like of the subject.

The input/output interface 204 functions as, for example, an interface of the CPU 201 with the input device 21 and the output device 22. The input/output interface 204 can include various communication modules communicating with an input device such as a mouse or a keyboard or a driving module driving the display 221 and the speaker 222.

FIG. 4 is a functional block diagram illustrating examples of main functions of the control unit 20. The control unit 20 functions as a measurement control unit 2011, an acquisition unit 2012, an image generation unit 2013, a first estimation unit 2014, a derivation unit 2015, and an output unit 2016.

The measurement control unit 2011 integrally controls the non-ultrasonic sensor 1 and the ultrasonic sensor 3 for simultaneous measurement using the non-ultrasonic sensor 1 and the ultrasonic sensor 3. The measurement control unit 2011 controls the non-ultrasonic sensor 1 and the ultrasonic sensor 3, for example, for measurement over time. For example, the measurement control unit 2011 controls a pressurization pump, an exhaust valve, and the like connected to the cuff and causes one ultrasonic element 30 or the plurality of ultrasonic elements 30 to send ultrasonic waves at a timing instructed by the user.

The acquisition unit 2012 acquires sensor information from each of the non-ultrasonic sensor 1 and the ultrasonic sensor 3. The acquisition unit 2012 acquires, for example, sensor information over time from each of the non-ultrasonic sensor 1 and the ultrasonic sensor 3. The acquisition unit 2012 acquires sensor information including information regarding a blood pressure of the subject from the non-ultrasonic sensor 1.

The acquisition unit 2012 acquires sensor information including ultrasonic wave information from the ultrasonic sensor 3. The ultrasonic wave information includes, for example, sending information and reception information. The sending information is information regarding an ultrasonic wave sent from the ultrasonic element 30 and includes, for example, information regarding a position of the driven ultrasonic element 30 and a driving voltage, a driving frequency, a waveform, a gain, a driving start time, a driving time, and the like of the ultrasonic element 30. The reception information is information regarding reflected waves reflected from blood vessels, bones, organs, and the like of the subject and received by the plurality of ultrasonic elements 30 and includes, for example, information regarding a frequency, a waveform, intensity, a reception time, and the like of the reflected waves. When ultrasonic waves are sent from one ultrasonic element 30, for example, the plurality of ultrasonic elements 30 receive the reflected waves.

The acquisition unit 2012 acquires at least the reception information from the ultrasonic sensor 3. For example, the acquisition unit 2012 acquires the reception information from each of the plurality of ultrasonic elements 30 via the network interface 23. The acquisition unit 2012 may acquire the sending information from the measurement control unit 2011.

The image generation unit 2013 generates ultrasonic image data of the subject based on the sensor information acquired from the ultrasonic sensor 3. For example, the image generation unit 2013 generates image data of a blood vessel of the subject. The image generation unit 2013 may generate image data using information directly acquired from the ultrasonic sensor 3. For example, the image generation unit 2013 generates image data such as an image of an M mode, an image of a B mode, an image of a color Doppler mode, or an image of a pulse Doppler mode by performing each process on the sensor information acquired from the ultrasonic sensor 3. The image generation unit 2013 may generate image data of a bone, an organ, or the like of the subject.

5. The first estimation unit 2014 estimates physiological information of the subject based on the sensor information acquired from the ultrasonic sensor 3. For example, the first estimation unit 2014 estimates a quantity (flow quantity) of blood flowing in a blood vessel of the subject. For example, the first estimation unit 2014 estimates a flow quantity of a blood vessel from an ultrasonic image generated by the image generation unit 2013. For example, the first estimation unit 2014 estimates a flow quantity of the blood vessel over time.

The derivation unit 2015 derives the predetermined parameter indicating a body state of the subject based on the sensor information acquired from each of the non-ultrasonic sensor 1 and the ultrasonic sensor 3. As will be described in detail below, by deriving the predetermined parameter using the sensor information acquired from each of the plurality of sensors as such, it is possible to ascertain a body state of a subject diversely.

For example, the derivation unit 2015 derives the predetermined parameter using the sensor information acquired from the non-ultrasonic sensor 1 and the physiological information estimated by the first estimation unit 2014. For example, the derivation unit 2015 derives the predetermined parameter over time. The sensor information acquired from the non-ultrasonic sensor 1 includes information regarding a blood pressure of the subject. When the first estimation unit 2014 estimates a flow quantity of a blood vessel of the subject, the derivation unit 2015 derives blood vessel resistance of the subject using, for example, a value of the blood pressure and a flow quantity of the blood vessel of the subject at the same time. The derivation unit 2015 derives blood vessel resistance of the subject, for example, using the following Formula (1).

$$\begin{matrix}[ Math . 1 ] & \\ R = P / I & ( 1 )\end{matrix}$$

In Formula (1), R is blood vessel resistance, P is a blood pressure, and I is a flow quantity.

The output unit 2016 outputs information regarding the predetermined parameter derived by the derivation unit 2015 to the output device 22 or the like. The output unit 2016 outputs the information regarding the predetermined parameter in association with the sensor information acquired from each of the non-ultrasonic sensor 1 and the ultrasonic sensor 3 to the output device 22 or the like. Accordingly, the user can easily compare the information directly acquired from each of the non-ultrasonic sensor 1 and the ultrasonic sensor 3 with the derived parameter, and thus can ascertain the body state of the subject more diversely.

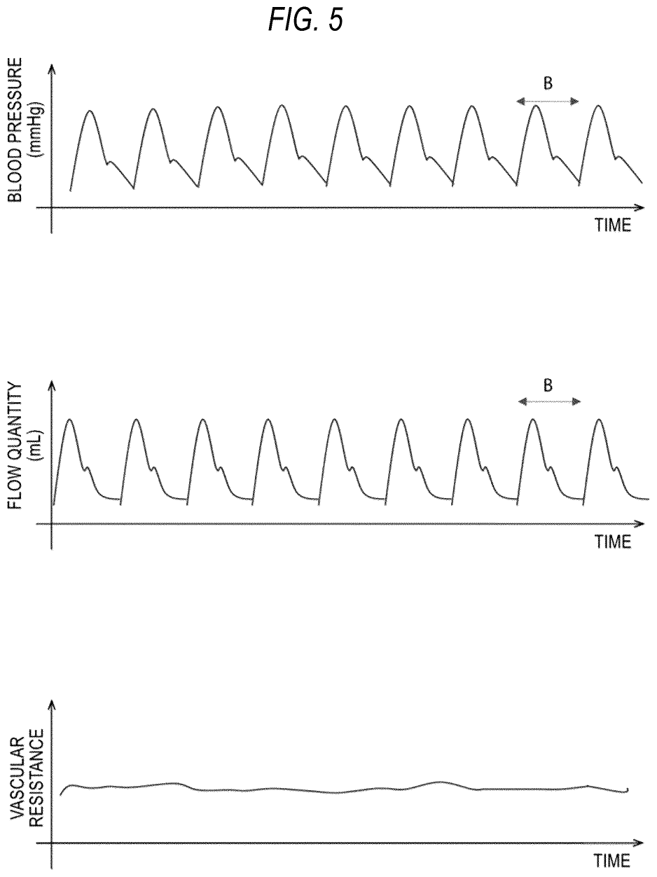

FIG. 5 illustrates an example of the information regarding the predetermined parameter displayed on the display 221. In FIG. 5, a change over time in the blood vessel resistance is illustrated along with changes over time in the blood pressure and a flow quantity of the blood vessel of the subject. Accordingly, the user can easily confirm the change over time in each of the blood pressure, the flow quantity of the blood vessel, and the blood vessel resistance of the subject and can perform diagnosis, treatment, or the like more accurately on the subject. The output unit 2016 may output an average value corresponding to one beat B or per unit time as the blood pressure and the flow quantity of the blood vessel of the subject.

The output unit 2016 may further display other physiological information measured for the subject on the display 221. The output unit 2016 may further display, for example, physiological information such as a heart rate and SpO2 measured for the subject on the display 221.

### (Processing Method of Information Processing Apparatus)

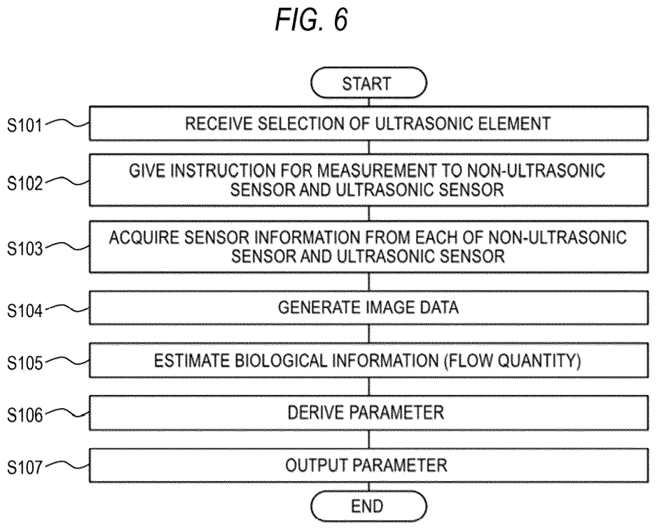

FIG. 6 is a flowchart illustrating an example of a process by the information processing apparatus 2. The flowchart can be performed, for example, according to a program stored in the information processing apparatus 2.

First, the information processing apparatus 2 receives a selection of the ultrasonic element 30 to send an ultrasonic wave (step S101). For example, when the user inputs a selection of the ultrasonic element 30 to the input device 21, the information processing apparatus 2 receives the selection of the ultrasonic element 30. The user can select the ultrasonic element 30, for example, while confirming an image of a blood vessel or the like of the subject displayed on the display 221.

Subsequently, the information processing apparatus 2 instructs each of the non-ultrasonic sensor 1 and the ultrasonic sensor 3 to perform measurement (step S102). Specifically, the pressurization pump, the exhaust valve, and the like are controlled such that the non-ultrasonic sensor 1 measures a blood pressure of the subject, and the ultrasonic element 30 selected in step S101 is instructed to send the ultrasonic wave so that the ultrasonic sensor 3 performs ultrasonic measurement on the subject.

Subsequently, the information processing apparatus 2 acquires the sensor information from each of the non-ultrasonic sensor 1 and the ultrasonic sensor 3 (step S103). For example, the information processing apparatus 2 acquires the sensor information including the information regarding the blood pressure of the subject from the non-ultrasonic sensor 1 and acquires the sensor information including the ultrasonic information from the ultrasonic sensor 3.

Subsequently, the information processing apparatus 2 generates image data of the blood vessel or the like of the subject based on the sensor information acquired from the ultrasonic sensor 3 (step S104). Subsequently, the information processing apparatus 2 estimates the physiological information of the subject based on the image data generated in step S104 (step S105). For example, the information processing apparatus 2 estimates a flow quantity of the blood vessel of the subject. The details of the step will be described below.

Subsequently, the information processing apparatus 2 derives the predetermined parameter based on the sensor information acquired from each of the non-ultrasonic sensor 1 and the ultrasonic sensor 3 in step S103 (step S106). For example, the information processing apparatus 2 derives blood vessel resistance of the subject using the sensor information acquired from the non-ultrasonic sensor 1 and the physiological information of the subject estimated in step S105. Thereafter, the information processing apparatus 2 outputs information regarding the derived predetermined parameter to the output device 22 or the like (step S107), and then ends the process.

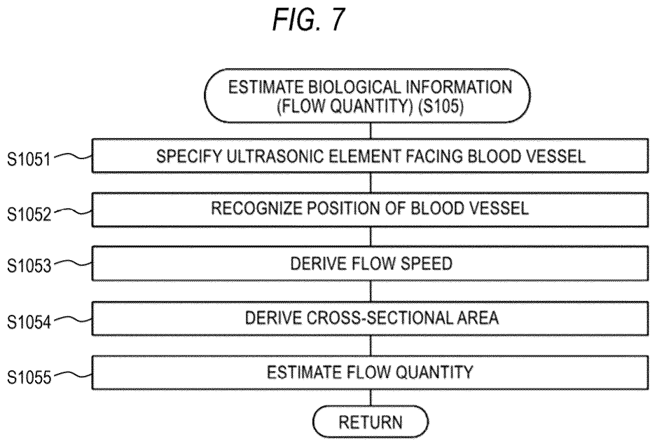

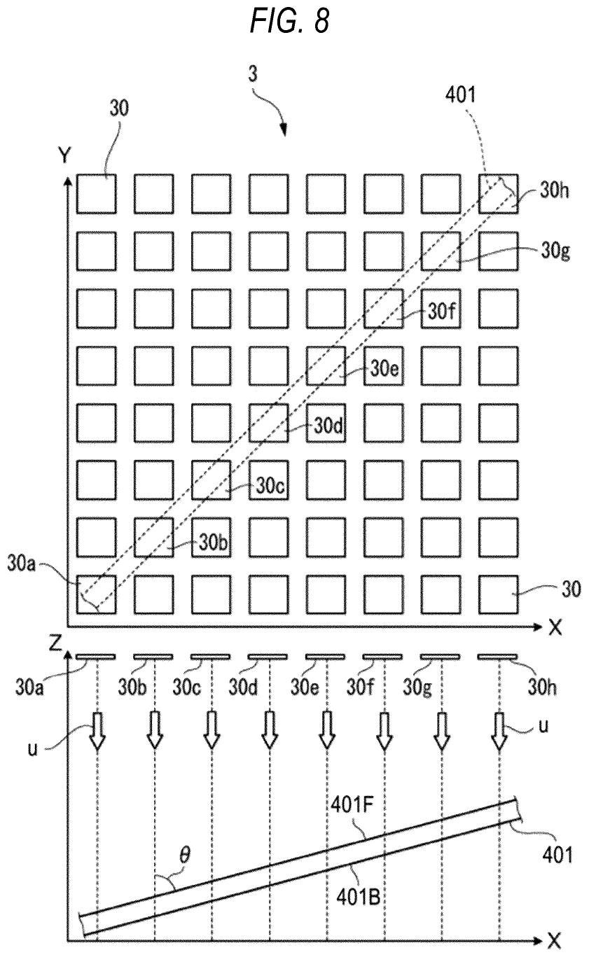

Here, the process of step S105 will be described with reference to FIGS. 7 and 8. Here, an example in which the information processing apparatus 2 estimates a flow rate of a blood vessel (blood vessel 401) of the subject as physiological information will be described. FIG. 7 is a flowchart of a sub-routine of step S105. FIG. 8 illustrates an example of a relation between the predetermined blood vessel 401 of the subject and the ultrasonic sensor 3 (the ultrasonic element 30). Hereinafter, arrangement directions of the ultrasonic elements 30 may be referred to as X and Y directions, and a direction orthogonal to the X and Y directions may be referred to as a Z direction. The ultrasonic element 30 transmits an ultrasonic wave u in directions intersecting the X and Y directions.

The information processing apparatus 2 first specifies the ultrasonic elements 30 facing the blood vessel 401 in the Z direction based on the sensor information acquired from the ultrasonic sensor 3 in the process of step S103 (step S1051). For example, ultrasonic elements 30a to 30h among the plurality of ultrasonic elements 30 face the blood vessel 401 (FIG. 8). The information processing apparatus 2 specifies, for example, the ultrasonic elements 30a to 30h facing the blood vessel 401 as follows.

Since the ultrasonic wave (ultrasonic wave u) is reflected from a boundary between media with different acoustic impedance, the ultrasonic wave is reflected in a boundary between the blood vessel 401 and a physiological tissue adjacent to the blood vessel 401. In particular, since a difference in acoustic impedance between the blood vessel 401 and a physiological tissue adjacent to the blood vessel 401 is large, the ultrasonic wave u is strongly reflected from a front side wall 401F and a rear side wall 401B of the blood vessel 401. With the strong reflection of the ultrasonic wave u, the information processing apparatus 2 can specify the ultrasonic elements 30a to 30h.

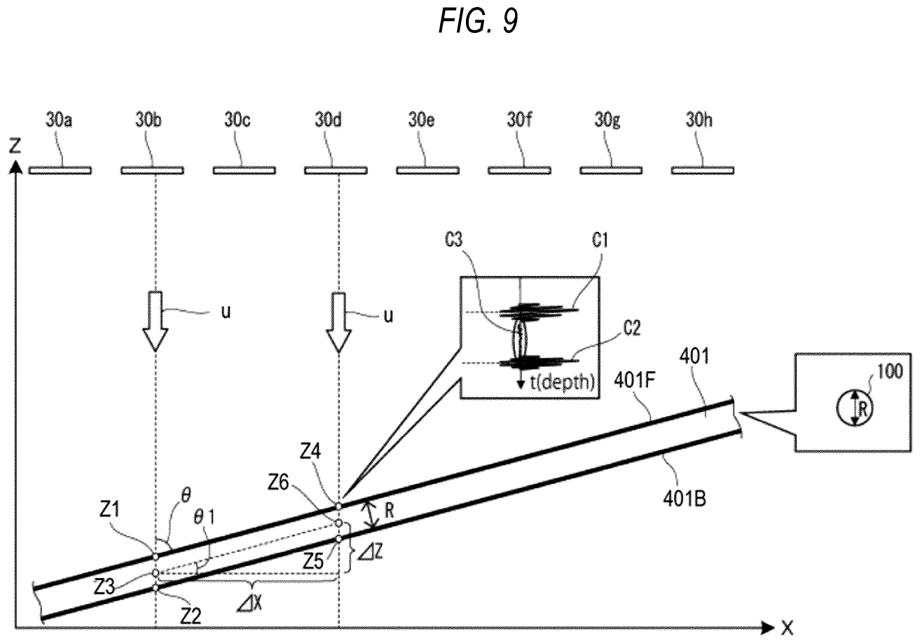

Alternatively, a pattern of the reflected wave of the ultrasonic wave u reflected from the front side wall 401F and the rear side wall 401B of the blood vessel 401 may be a known pattern. The information processing apparatus 2 may specify the ultrasonic elements 30 (the ultrasonic elements 30a to 30h) acquiring predetermined reception information (for example, reflected waves C1 and C2 or the like illustrated in FIG. 9 to be described below). The information processing apparatus 2 specifies the ultrasonic elements 30a to 30h facing the blood vessel 401, and then recognizes a position of the blood vessel 401 (step S1052).

FIG. 9 illustrates an example of a relation between the ultrasonic elements 30a to 30h and the blood vessel 401 in the X and Z directions. The information processing apparatus 2 recognizes the position of the blood vessel 401 by determining positions Z1 to Z2 of the blood vessel 401 facing two ultrasonic elements 30 (for example, ultrasonic elements 30b and 30d) among the ultrasonic elements 30a to 30h.

The positions Z1 and Z2 are positions of the front side wall 401F and the rear side wall 401B of the blood vessel 401 in the Z direction facing the ultrasonic element 30b, respectively, and the position Z3 is a middle position between the positions Z1 and Z2. The positions 24 and 25 are positions of the front side wall 401F and the rear side wall 401B of the blood vessel 401 in the Z direction facing the ultrasonic element 30d, respectively, and the position Z6 is a middle position between the positions Z4 and Z5.

For example, the information processing apparatus 2 can determine the position Z1 as follows. The information processing apparatus 2 first specifies a time (time t0) at which the ultrasonic wave u is sent from the ultrasonic element 30b to the blood vessel 401 and a time (time t4) at which the ultrasonic wave u is reflected from the front side wall 401F and is received by the ultrasonic element 30b based on the sensor information acquired from the ultrasonic sensor 3. Subsequently, the information processing apparatus 2 determines a distance L1 between the ultrasonic element 30b and the front side wall 401F in the Z direction based on times 10 and 14. The distance L1 can be determined using the following Formula (2). The information processing apparatus 2 determines the position Z1 according to the distance L1 between the ultrasonic element 30b and the front side wall 401F.

$$\begin{matrix}[ Math . 2 ] & \\ L 1 = c ( t 4 - t 0 ) / 2 & ( 2 )\end{matrix}$$

Here, c is a sound velocity of the ultrasonic wave u traveling inside a physiological tissue.

The information processing apparatus 2 determines the position Z2 as in the position. Z1. Specifically, the information processing apparatus 2 first specifies the time (time t0) at which the ultrasonic wave u is sent from the ultrasonic element 30b to the blood vessel 401 and a time (time 15) at which the ultrasonic wave u is reflected from the rear side wall 401B and is received by the ultrasonic element 30b based on the sensor information acquired from the ultrasonic sensor 3. Subsequently, the information processing apparatus 2 determines a distance L2 between the ultrasonic element 30b and the rear side wall 401B in the Z direction based on times 10 and 15. The distance L2 can be determined using the following Formula (3). The information processing apparatus 2 determines the position Z2 according to the distance L2 between the ultrasonic element 30b and the rear side wall 401B.

$$\begin{matrix}[ Math . 3 ] & \\ L 2 = c ( t 5 - t 0 ) / 2 & ( 3 )\end{matrix}$$

Here, c is a sound velocity of the ultrasonic wave u traveling inside a physiological tissue.

After the positions Z1 and Z2 are determined, the information processing apparatus 2 determines the position Z3 which is a middle position between the positions Z1 and Z2 by the following Formula (4).

$$\begin{matrix}[ Math . 4 ] & \\ Z 3 = ( Z 1 + Z 2 ) / 2 & ( 4 )\end{matrix}$$

The information processing apparatus 2 determines the positions Z4 to 26 in the Z direction facing the ultrasonic element 30d as in the positions Z1 to Z3, and recognizes the position of the blood vessel 401.

The information processing apparatus 2 recognizes the position of the blood vessel 401, and then calculates a flow rate of the blood vessel 401 (step S1053). The information processing apparatus 2 calculates a flow rate of the blood vessel 401, for example, using an angle formed by the blood vessel 401 with respect to the Z direction and a Doppler frequency shift.

For example, the information processing apparatus 2 determines an angle θ formed by the blood vessel 401 with respect to the Z direction as follows. The information processing apparatus 2 first determines an angle (an angle θ1) formed between the blood vessel 401 with respect to the X direction based on a distance ΔZ between the positions 26 and Z3 in the Z direction and a distance ΔX between the ultrasonic elements 30b and 30d in the X direction. The angle θ1 is determined using, for example, the following Formula (5).

$$\begin{matrix}[ Math . 5 ] & \\ θ 1 = {tan}^{- 1} ( Δ Z / Δ X ) & ( 5 )\end{matrix}$$

Here, the angles θ and θ1 have a relation of the following Formula (6). Accordingly, the information processing apparatus 2 can determines the angle θ.

$$\begin{matrix}[ Math . 6 ] & \\ θ = ( 90 ° - θ 1 ) & ( 6 )\end{matrix}$$

The information processing apparatus 2 determines an angle formed by the blood vessel 401 with respect to the Z direction, and then calculates a Doppler frequency shift in the blood vessel 401.

When v is a velocity (flow rate) of blood flowing in the blood vessel 401, erythrocyte included in the blood moves at a velocity v in the blood vessel 401. The erythrocyte is observed to be moving at a velocity of vcosθ in the Z direction. In the ultrasonic wave u reflected by the erythrocyte, a frequency is changed by the Doppler effect.

When f₀ is a frequency of the ultrasonic wave u transmitted from the ultrasonic element 30, f_{d} is a Doppler frequency shift, v is a velocity of blood flowing in the blood vessel 401, and c is a sound velocity of the ultrasonic wave u traveling inside a physiological tissue, the Doppler frequency shift f_{d} is derived by the following Formula (7).

$$\begin{matrix}[ Math . 7 ] & \\ {f}_{d} = 2 {vf}_{0} \times cos θ / c & ( 7 )\end{matrix}$$

From the foregoing Formula (7), it is understood that the Doppler frequency shift f_{d} changes in proportion to the velocity v of the blood flowing in the blood vessel 401. In other words, by measuring the Doppler frequency shift f_{d}, it is possible to measure the velocity v (flow rate).

The information processing apparatus 2 can calculate the Doppler frequency shift f_{d}, for example, using a pulse Doppler method or a color Doppler method. The Doppler frequency shift f_{d} is calculated using the pulse Doppler method as follows, for example. The information processing apparatus 2 first acquires data indicating a change in a sum of luminance values of the reflected waves C3 (see FIG. 9) of the ultrasonic wave u reflected by the erythrocyte in the blood vessel 401. Thereafter, the information processing apparatus 2 calculates the Doppler frequency shift f_{d} by performing fast Fourier transform (FFT) processing on the acquired data.

When the color Doppler method is used, the information processing apparatus 2 calculates the Doppler frequency shift f_{d} according to, for example, an autocorrelation method. When a frequency of the ultrasonic waves is 8 kHz (in other words, 8000 ultrasonic pulses are output for 1 second), the information processing apparatus 2 may calculate the Doppler frequency shift f_{d} for every 140 ultrasonic pulses. Here, an update rate of the Doppler frequency shift f_{d} is 57 Hz.

In the pulse Doppler method, a calculation load by the FFT is relatively large. Therefore, in the calculation of the Doppler frequency shift f_{d}, it is preferable to use a color Doppler method with a relatively low calculation load.

The information processing apparatus 2 calculates the velocity v (flow rate) of the blood flowing in the blood vessel 401 by Formula (7) using the angle θ and the Doppler frequency shift f_{d} obtained as above.

The information processing apparatus 2 calculates the flow rate of the blood vessel 401, and then calculates a cross-sectional area of the blood vessel 401 (step S1054). The cross-sectional area is a cross-sectional area perpendicular in an axial direction of the blood vessel 401 and is a cross-sectional area of a hollow portion of the blood vessel 401 in which the blood flows.

For example, a cross-sectional area S of the blood vessel 401 is obtained using an inner diameter R of the blood vessel 401 by the following Formula (8).

$$\begin{matrix}[ Math . 8 ] & \\ S = π {R}^{2} / 4 & ( 8 )\end{matrix}$$

Since a distance between the positions Z1 and Z2 is (Z1−Z2|, the inner diameter R is expressed as in the following Formula (9). The inner diameter R may be calculated using a distance |Z3−Z4| between the positions Z3 and Z4.

$$\begin{matrix}[ Math . 9 ] & \\ R = \vert Z 1 - Z 2 \vert sin θ & ( 9 )\end{matrix}$$

The information processing apparatus 2 calculates the cross-sectional area S of the blood vessel 401 by, for example, Formulae (8) and (9).

The information processing apparatus 2 calculates the cross-sectional area of the blood vessel 401, and then estimates a flow quantity of the blood vessel 401 (step S1055). A flow quantity Q of the blood vessel 401 can be estimated using, for example, the cross-sectional area S and the velocity v of the blood flowing in the blood vessel 401 by the following Formula (10).

$$\begin{matrix}[ Math . 10 ] & \\ Q = Sv & ( 10 )\end{matrix}$$

As such, the information processing apparatus 2 recognizes the position of the blood vessel 401, then calculates the flow rate and the cross-sectional area of the blood vessel 401, and estimates a flow quantity of the blood vessel 401 using the flow rate and the cross-sectional area

### (Operational Effects of Information Processing Apparatus and Information Processing System)

In the information processing apparatus 2 and the information processing system 100 according to the embodiment, a predetermined parameter indicating a body state of the subject is derived based on the sensor information acquired from each of the non-ultrasonic sensor 1 and the ultrasonic sensor 3. Accordingly, the user can ascertain the body state of the subject diversely. Hereinafter, the operational effects will be described.

An ultrasonic technique is used for image diagnosis or the like. However, it is difficult to determine a progress state of a disease and magnitude of a cure effect of the subject by only one vital parameter. A medical practitioner such as a doctor is required to determine the progress state of the disease and the magnitude of the cure effect of the subject while measuring various vital parameters of the subject using a plurality of sensors and investigating such complex interaction. Therefore, there is a concern that an accuracy of determination is affected by knowledge, experience, or the like of a medical practitioner.

In the information processing system 100 and the information processing apparatus 2, however, a parameter indicating a body state of a subject is derived based on the sensor information acquired from each of the non-ultrasonic sensor 1 and the ultrasonic sensor 3. For example, in the information processing system 100 and the information processing apparatus 2, blood vessel resistance is derived from a value of a blood pressure of the subject acquired from the non-ultrasonic sensor 1 and a flow quantity of a blood vessel of the subject acquired from the ultrasonic sensor 3. In other words, a correlation between a blood pressure and a flow quantity of the blood vessel of the subject is derived. Accordingly, the correlation between the blood pressure and the flow quantity of the blood vessel is ascertained more easily than when each of the blood pressure and the flow quantity of the blood vessel is separately measured. Accordingly, the user can ascertain a body state of the subject diversely regardless of knowledge, experience, or the like. Accordingly, the user can determine the progress state of the disease of the subject and the magnitude of the cure effect more accurately.

The derived parameter is a parameter which cannot be obtained from measurement by a single sensor, that is, a new parameter, and a state of the subject is easily ascertained more accurately than in measurement by only a single sensor.

For example, the parameter can be displayed on the display 221 along with the sensor information acquired from each of the non-ultrasonic sensor 1 and the ultrasonic sensor 3. In particular, by displaying the derived parameter and the sensor information acquired from each of the non-ultrasonic sensor 1 and the ultrasonic sensor 3 on the same screen, it is easier to determine the state of the subject comprehensively. For example, by displaying all of the blood pressure and the flow quantity of the blood vessel acquired from each of the non-ultrasonic sensor 1 and the ultrasonic sensor 3 and the blood vessel resistance derived therefrom as numerical information on the same screen, it is easy to determine the state of the subject comprehensively even when the display 221 is small.

The information processing system 100 and the information processing apparatus 2 preferably derive the predetermined parameter over time. Accordingly, the user can easily confirm a change in the predetermined parameter over time and easily ascertain a change in the body of the subject.

Hereinafter, modification examples and other embodiments of the information processing system 100 described in the foregoing first embodiment will be described. Hereinafter, to avoid repeated description, detailed description of the same configuration as each configuration of the information processing system 100 described in the foregoing first embodiment will be omitted,

### Modification Example

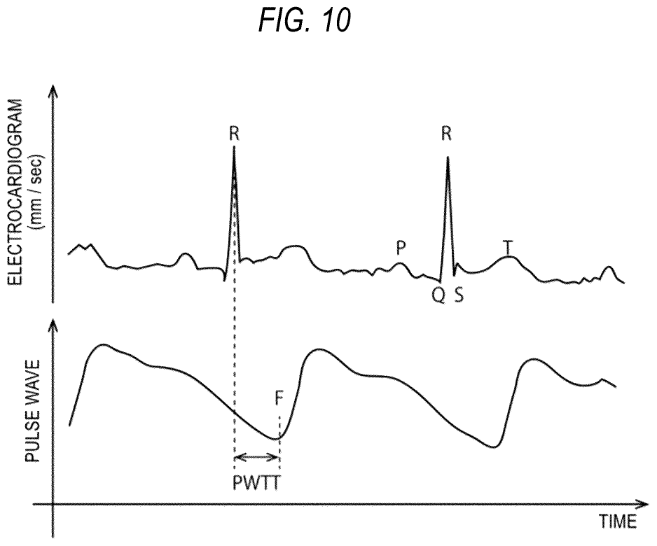

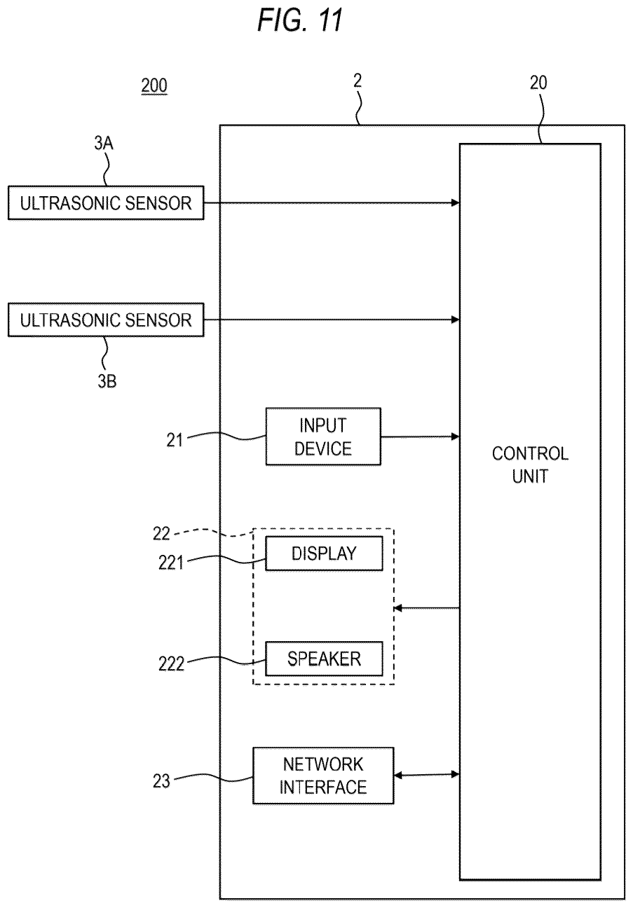

FIG. 10 illustrates an example of parameters derived by the information processing system 100 according to Modification Example 1. The information processing system 100 derives a pulse wave transit time (PWTT) based on the sensor information acquired from each of the non-ultrasonic sensor 1 and the ultrasonic sensor 3. In other words, a predetermined parameter derived by the non-ultrasonic sensor 1 and the ultrasonic sensor 3 is a pulse wave transit time. Except for such point, the information processing system 100 has a similar configuration to the information processing system 100 described in the foregoing embodiment, and similar operational effects are obtained.

The non-ultrasonic sensor 1 includes, for example, an electrode to measure electrocardiogram (ECG) of the subject. The sensor information acquired from the non-ultrasonic sensor 1 includes information regarding electrocardiogram.

The information processing apparatus 2 functions as the measurement control unit 2011, the acquisition unit 2012, the first estimation unit 2014, the derivation unit 2015, and the output unit 2016 (FIG. 4). The measurement control unit 2011 and the acquisition unit 2012 function similarly as described in the foregoing first embodiment. The information processing apparatus 2 may further function as the image generation unit 2013.

The first estimation unit 2014 estimates physiological information of the subject based on the sensor information acquired from the ultrasonic sensor 3. The first estimation unit 2014 estimates, for example, a pulse-wave waveform of the subject. For example, the ultrasonic sensor 3 receives waves generated by arteria pulses and propagating in the body of the subject, and thus the first estimation unit 2014 estimates the pulse-wave waveform of the subject. The first estimation unit 2014 may estimate a pulse-wave waveform by tracing a change in a diameter of a blood vessel from an image of a cross-section of the blood vessel of the subject generated by the sensor information acquired from the ultrasonic sensor 3. The first estimation unit 2014 estimates, for example, a pulse-wave waveform over time.

The derivation unit 2015 derives a pulse wave transit time of the subject based on the sensor information acquired from each of the non-ultrasonic sensor 1 and the ultrasonic sensor 3. The derivation unit 2015 derives a time from an R wave to a rise F of a pulse wave of electrocardiogram, for example, when the sensor information acquired from the non-ultrasonic sensor 1 includes information regarding the electrocardiogram of the subject and the first estimation unit 2014 estimates the pulse-wave waveform of the subject (FIG. 10).

The output unit 2016 outputs information regarding the pulse wave transit time derived by the derivation unit 2015 to the output device 22 or the like. The output unit 2016 may display the pulse wave transit time along with the electrocardiogram and the pulse-wave waveform of the subject on the display 221 (FIG. 10) and may display a diagnosis result or the like of the state of the subject using the derived pulse wave transit time (not illustrated).

The output unit 2016 may output another parameter calculated using the pulse wave transit time. For example, the output unit 2016 may output a cardiac output of the subject using the pulse wave transit time. A cardiac output CO of the subject can be calculated using, for example, a pulse wave transit time PWTT by the following Formula (11). A heart rate HR can be measured using, for example, the non-ultrasonic sensor 1. The heart rate HR may be calculated, for example, by counting the number of R waves per minute included in the electrocardiogram detected by the non-ultrasonic sensor 1.

$$\begin{matrix}[ Math . 11 ] & \\ CP = ( α K \times PWTT + β K ) \times HR & ( 11 )\end{matrix}$$

In Formula (11), CO is a cardiac output, PWTT is a pulse-wave transit time, HR is a heart rate, and α, β, and K are coefficients specific to the subject.

In the information processing system 100 according to Modification Example 1, as described in the foregoing first embodiment, a predetermined parameter indicating a body state of the subject is derived based on the sensor information acquired from each of the non-ultrasonic sensor 1 and the ultrasonic sensor 3. Accordingly, the user can ascertain the body state of the subject diversely.

The pulse wave transit time derived by the information processing system 100 is a useful parameter used for evaluation of measurement of arteriosclerosis, prediction of a cardiac output, measurement of an autonomic nervous function, or the like. The user can determine a body state of the subject more accurately using the pulse wave transit time of the subject.

Here, since the pulse-wave waveform is estimated using the sensor information acquired from the ultrasonic sensor 3, the wave-form waveform of the subject in various states can be easily estimated. Hereinafter, the operational effects will be described.

For example, it is considered that a pulse-wave waveform of plethysmogram using an SpO2 probe or the like is used, but it is difficult to measure a pulse-wave waveform of a fingertip of the subject due to various causes. For example, when a symptom of a circulation imperfection of the whole body is shown in the subject, a peripheral artery is closed. Therefore, it is difficult to measure a pulse-wave waveform of a fingertip. A state of the peripheral blood vessel is easily changed due to ambient temperature and accuracy of measurement of the pulse wave waveform at the fingertip decreases in some cases.

On the other hand, the ultrasonic sensor 3 can be attached to, for example, an upper arm, a centrum part, or the like of the subject to perform measurement. Accordingly, even when it is difficult to measure a pulse-wave waveform at a fingertip of the subject, the pulse-wave waveform of the subject can be easily estimated.

### Modification Example 2

The information processing system 100 according to Modification Example 2 derives an oxygen transport rate based on the sensor information acquired from each of the non-ultrasonic sensor 1 and the ultrasonic sensor 3. In other words, a predetermined parameter derived by the information processing system 100 is an oxygen transport rate. The oxygen transport rate is, for example, an amount of oxygen arriving at a peripheral tissue. Except for such point, the information processing system 100 has a similar configuration to the information processing system 100 described in the foregoing embodiment, and similar operational effects are obtained.

The non-ultrasonic sensor 1 includes, for example, an SpO2 probe to measure arterial oxygen saturation of the subject. The sensor information acquired from the non-ultrasonic sensor 1 includes information regarding arterial oxygen saturation.

The information processing apparatus 2 functions as the measurement control unit 2011, the acquisition unit 2012, the image generation unit 2013, the first estimation unit 2014, the derivation unit 2015, and the output unit 2016 (FIG. 4). The measurement control unit 2011, the acquisition unit 2012, and the image generation unit 2013 function similarly as described in the foregoing first embodiment.

The first estimation unit 2014 estimates physiological information of the subject based on the sensor information acquired from the ultrasonic sensor 3. The first estimation unit 2014 estimates, for example, a cardiac output of the subject. The first estimation unit 2014 performs image analysis of an image of the vicinity of a heart generated by the image generation unit 2013 and estimates the cardiac output based on a motion in the vicinity of the heart. Specifically, the cardiac output is estimated by obtaining an amount of blood output from an artery or a change amount of a volume of the heart through the image analysis. For example, the first estimation unit 2014 estimates the cardiac output over time.

The derivation unit 2015 derives an oxygen transport rate of the subject based on the sensor information acquired from each of the non-ultrasonic sensor 1 and the ultrasonic sensor 3. The derivation unit 2015 derives an oxygen transport rate of arterial blood using, for example, the following Formulae (12) and (13), for example, when the sensor information acquired from the non-ultrasonic sensor 1 includes information regarding the arterial oxygen saturation of the subject and the first estimation unit 2014 estimates the cardiac output of the subject. A hemoglobin concentration Hb can be obtained, for example, by performing blood gas measurement of the subject and is input by the user via the input device 21. The information processing system 100 may include a blood gas measurement device and the acquisition unit 2012 may acquire information regarding the hemoglobin concentration Hb of the subject from the blood gas measurement device.

$$\begin{matrix}[ Math . 12 ] & \\ D O 2 = CaO 2 \times CO & ( 12 )\end{matrix}$$

$$CaO 2 \approx 1.34 \times Hb \times SpO 2 \qquad ( 13 )$$

In Formulae (12) and (13), DO2 indicates an oxygen transport rate of arterial blood, CaO2 indicates an oxygen content contained in blood per unit amount, CO indicates a cardiac output, Hb indicates a hemoglobin concentration per unit blood flow, and SpO2 indicates an arterial oxygen saturation.

The output unit 2016 outputs information regarding the oxygen transport rate derived by the derivation unit 2015 to the output device 22 or the like. The output unit 2016 may display the oxygen transport rate along with the arterial oxygen saturation and the cardiac output of the subject on the display 221 and may display a diagnosis result or the like of a state of the subject using the derived oxygen transport rate.

In the information processing system 100 according to Modification Example 2, as described in the foregoing first embodiment, a predetermined parameter indicating a body state of the subject is derived based on the sensor information acquired from each of the non-ultrasonic sensor 1 and the ultrasonic sensor 3. Accordingly, the user can ascertain the body state of the subject diversely

In Modification Example 2, the example in which the oxygen transport rate is an amount of oxygen arriving at a peripheral tissue has been described, but the oxygen transport rate may be an amount of oxygen arriving at a predetermined organ. Then, the first estimation unit 2014 estimates a flow quantity of a predetermined blood vessel, such as a flow quantity of an internal carotid artery, for example, based on the sensor information acquired from the ultrasonic sensor 3.

### Second Embodiment

FIG. 11 is a block diagram illustrating an example of an overall hardware configuration of the information processing system 200 according to a second embodiment. The information processing system 200 includes the information processing apparatus 2 and ultrasonic sensors 3A and 3B (first and second ultrasonic sensors). The ultrasonic sensors 3A and 3B can each be connected to the information processing apparatus 2. The information processing system 200 according to the second embodiment includes a plurality of ultrasonic sensors (ultrasonic sensors 3A and 3B). Except for such point, the information processing system 200 according to the second embodiment has a similar configuration as the information processing system 100 described in the foregoing first embodiment and similar operational effects are obtained.

For example, the ultrasonic sensors 3A and 3B have similar configuration as that of the ultrasonic sensor 3 described in the foregoing first embodiment (FIGS. 2(A) to 2(C)) and are attached to a body surface of the subject. The ultrasonic sensors 3A and 3B are attached to, for example, the vicinity of a urinary bladder.

The information processing apparatus 2 acquires sensor information from each of the ultrasonic sensors 3A and 3B. The information processing apparatus 2 includes, for example, as described in the foregoing first embodiment, the control unit 20, the input device 21, the output device 22, and the network interface 23.

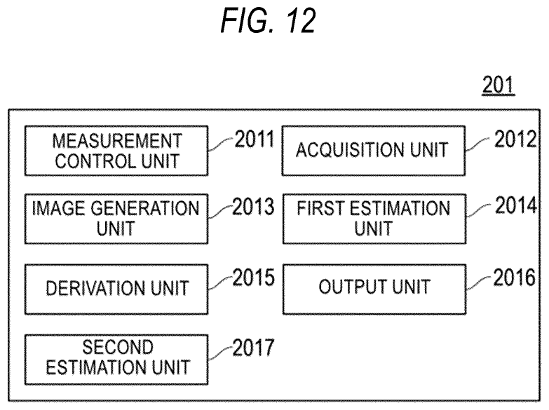

FIG. 12 is a functional block diagram illustrating main functions of the control unit 20. The control unit 20 functions as the measurement control unit 2011, the acquisition unit 2012, the image generation unit 2013, the first estimation unit 2014, the derivation unit 2015, the output unit 2016, and a second estimation unit 2017. For example, the measurement control unit 2011, the acquisition unit 2012, and the image generation unit 2013 function similarly as described in the foregoing first embodiment.

The first estimation unit 2014 estimates first physiological information of the subject based on the sensor information acquired from the ultrasonic sensor 3A. For example, the first estimation unit 2014 estimates, for example, a flow quantity of a blood vessel of the subject, more specifically, a renal blood flow rate which is a flow quantity of blood in a kidney. For example, the first estimation unit 2014 causes the image generation unit 2013 to generate an image of the kidney and estimates the renal blood flow rate. For example, the first estimation unit 2014 estimates the renal blood flow rate over time.

The second estimation unit 2017 estimates second physiological information of the subject based on the sensor information acquired from the ultrasonic sensor 3B. The second physiological information is physiological information different from the first physiological information. For example, the second estimation unit 2017 estimates a urinary volume change rate per unit time. For example, the second estimation unit 2017 performs image analysis of an image of the vicinity of the urinary bladder generated by the image generation unit 2013 and estimates the urinary volume change rate based on a difference between luminance values of the inside and the outside of the urinary bladder. A tissue on the outside of the urinary bladder reflects an ultrasonic wave and a tissue on the inside of the urinary bladder is a homogeneous medium and transmits the ultrasonic wave. Accordingly, an extreme difference in luminance occurs between the inside and the outside of the urinary bladder, and the urinary volume change rate can be estimated. For example, the second estimation unit 2017 estimates the urinary volume change rate over time.

The derivation unit 2015 derives a predetermined parameter indicating a body state of the subject based on the sensor information acquired from each of the ultrasonic sensors 3A and 3B. For example, the derivation unit 2015 derives the predetermined parameter using the first physiological information and the second physiological information estimated by the first estimation unit 2014 and the second estimation unit 2017. When the first estimation unit 2014 estimates the renal blood flow rate of the subject and the second estimation unit 2017 estimates the urinary volume change rate of the subject, the derivation unit 2015 derives, for example, a glomerular filtration rate (GFR) of the subject using the renal blood flow rate and the urinary volume change rate of the subject at the same time. The glomerular filtration rate is an index for measuring performance for producing urine by filtering blood made by a glomerulus of a kidney for 1 minute and is an important index used for a doctor or the like to determine a kidney function state or a dosage.

The output unit 2016 outputs information regarding the glomerular filtration rate of the subject derived by the derivation unit 2015 to the output device 22 or the like. For example, the output unit 2016 outputs the information regarding the glomerular filtration rate in association with the sensor information acquired from each of the ultrasonic sensors 3A and 3B to the output device 22 or the like.

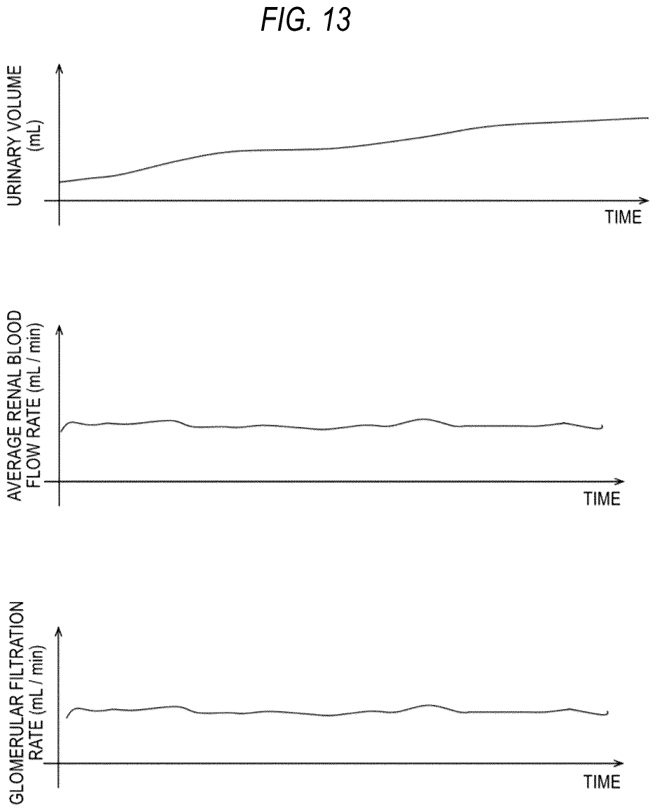

FIG. 13 illustrates an example of the information of the glomerular filtration rate displayed on the display 221 or the like. In FIG. 13, the glomerular filtration rate over time is illustrated along with an average renal blood flow rate (mL/min) and a urinary volume (mL) of the subject. Accordingly, the user can easily confirm a change in each of the average renal blood flow rate, the urinary volume, and the glomerular filtration rate over time.

In the information processing system 200 according to the second embodiment, a predetermined parameter indicating a body state of the subject is derived based on the sensor information acquired from each of the ultrasonic sensors 3A and 3B. Accordingly, the user can ascertain the body state of the subject diversely.

In the information processing system 200, the glomerular filtration rate of the subject can be derived. The glomerular filtration rate can also be estimated from inulin detection and a creatinine value by blood examination. Then, a long time is necessary in the examination. In the information processing system 200, however, the glomerular filtration rate of the subject can be derived more simply using the ultrasonic sensors 3A and 3B.

As described above, in the embodiments and the modification examples, the information processing apparatus and the information processing system according to the presently disclosed subject matter have been described. However, the presently disclosed subject matter can be appropriately added, modified, omitted by those skilled in the art within the scope of the technical spirit of the presently disclosed subject matter.

For example, in the foregoing embodiments or the like, the examples in which the information processing systems 100 and 200 can include two sensors (the non-ultrasonic sensor 1 and the ultrasonic sensor 3 or the ultrasonic sensors 3A and 3B) have been described, but the information processing system may include three sensors or more including an ultrasonic sensor.

In the foregoing embodiments and the like, the examples in which the sensor information acquired from the non-ultrasonic sensor 1 is information regarding a blood pressure, information regarding electrocardiogram, or information regarding arterial oxygen saturation have been described, but the sensor information acquired from the non-ultrasonic sensor 1 may include two or more pieces of information from the above information. Alternatively, the sensor information acquired from the non-ultrasonic sensor 1 may be another piece of information detected from the body of the subject.

In the foregoing embodiments and the like, the examples in which the information processing apparatus 2 derives the blood vessel resistance, the pulse wave transit time, the oxygen transport rate, or the glomerular filtration rate of the subject have been described, but the information processing apparatus 2 may derive another parameter indicating a body state of the subject. Alternatively, the information processing apparatus 2 may derive a plurality of parameters indicating a body state of the subject.

In the foregoing embodiments and the like, the examples in which the first estimation unit 2014 estimates a flow volume of a blood vessel, a pulse-wave waveform, or a cardiac output of the subject have been described, but the first estimation unit 2014 may estimate another piece of physiological information such as a cross-sectional area or a flow rate of a blood vessel of the subject or may estimate two pieces or more of physiological information.

In the foregoing second embodiment, the example in which the second estimation unit 2017 estimates a urinary volume change rate of the subject has been described, but the second estimation unit 2017 may estimate another piece of physiological information such as a cross-sectional area, a flow rate, a flow quantity, a pulse-wave waveform, or a cardiac output of a blood vessel of the subject or may estimate two pieces or more of physiological information.

In the foregoing embodiments and the like, the example in which the information processing apparatus 2 estimates a flow quantity of a blood vessel using the angle θ and the Doppler frequency shift f_{d} has been described, but the flow quantity of the blood vessel may be estimated by another method.

In the foregoing embodiments and the like, the example in which the ultrasonic sensor 3, 3A, or 3B is attached to a body surface of the subject for use has been described. The ultrasonic sensor 3, 3A, or 3B may not include the sheet-shaped member 32 or may not be attached for use. For example, the ultrasonic sensor 3, 3A, or 3B may include a so-called rigid type probe.

In the foregoing embodiments and the like, the example in which the ultrasonic elements 30 are provided at constant intervals (intervals a) in the ultrasonic sensor 3 has been described, but intervals of adjacent ultrasonic elements 30 may differ depending on the position of the ultrasonic sensor 3.

In the foregoing embodiments and the like, the example in which the information processing apparatus 2 generates image data of a blood vessel or the like of the subject has been described, but the information processing apparatus 2 may derive a predetermined parameter without generating image data.

Means and methods for performing various processes in the information processing apparatus 2 according to the above-described embodiments can also be implemented by any of a dedicated hardware circuit or a computer with a program. The program may be provided by a computer-readable recording medium such as a compact disc read only memory (CD-ROM) or may be provided online via a network such as the Internet. Here, the program recorded in the computer-readable recording medium is normally transmitted and stored in a memory such as a hard disk. The program may be provided as single application software or may be embedded in software of the information processing apparatus 2 as a function of the apparatus. 30

Units of processes in the flowchart in the foregoing embodiment are divided according to main processing contents to facilitate understanding of each process. The presently disclosed subject matter is not limited by a method of dividing the processing steps. Each process can be further divided into more processing steps. In one processing step, a plurality of processes may be performed.

### REFERENCE SIGNS LIST

100,200 Information processing system
1 Non-ultrasonic sensor
2 Information processing apparatus
20 Control unit
201 CPU
202 Memory
203 Auxiliary memory
204 Input/output interface
2011 Measurement control unit
2012 Acquisition unit
2013 Image generation unit
2014 First estimation unit
2015 Derivation unit
2016 Output unit
2017 Second estimation unit
21 Input device
22 Output device
23 Network interface
3, 3A, 3B Ultrasonic sensor
30 Ultrasonic element
32 Sheet-shaped member
34 Cable
35 Connection member

1. An information processing apparatus comprising:
a controller configured to:
acquire sensor information from each of a plurality of sensors including an ultrasonic sensor coming into contact with or in proximity to a subject; and
derive a predetermined parameter indicating a body state of the subject based on the acquired sensor information.

2. The information processing apparatus according to claim 1, wherein
the controller is configured to acquires the sensor information over time from each of the plurality of sensors, and
the controller is configured to derives the parameter over time.

3. The information processing apparatus according to claim 1, wherein:
the controller is configured to estimate physiological information of the subject based on the sensor information acquired from the ultrasonic sensor, and
the controller is configured to derives the parameter using the estimated physiological information.

4. The information processing apparatus according to claim 3, wherein the physiological information includes at least one of a flow quantity of a blood vessel, a pulse-wave waveform, and a cardiac output of the subject.

5. The information processing apparatus according to claim 1, wherein the plurality of sensors includes the ultrasonic sensor and a non-ultrasonic sensor detecting physical information other than an ultrasonic wave.

6. The information processing apparatus according to claim 5, wherein the sensor information acquired from the non-ultrasonic sensor includes at least one of information regarding a blood pressure, information regarding electrocardiogram, and information regarding an arterial oxygen saturation.

7. The information processing apparatus according to claim 6, wherein the derived parameter includes at least one of blood vessel resistance, a pulse wave transit time, and an oxygen transport rate.

8. The information processing apparatus according to claim 1, wherein the plurality of sensors includes first and second ultrasonic sensors.

9. The information processing apparatus according to claim 8, wherein:
the controller is configured to:
estimate first physiological information of the subject based on the sensor information acquired from the first ultrasonic sensor; and
estimate second physiological information of the subject based on the sensor information acquired from the second ultrasonic sensor, and
the controller is configured to derives the parameter using the estimated first physiological information and the estimated second physiological information.

10. The information processing apparatus according to claim 9, wherein
the first physiological information of the subject includes a renal blood flow quantity of the subject, and
the second physiological information of the subject includes a urinary volume change rate of the subject.

11. The information processing apparatus according to claim 10, wherein the derived parameter includes a glomerular filtration rate.

12. An information processing system comprising:
a plurality of sensors including an ultrasonic sensor; and
the information processing apparatus according to claim 1.

13. A non-transitory computer readable storage medium storing an information processing program causing a computer to perform processes of:
acquiring sensor information from each of a plurality of sensors including an ultrasonic sensor coming into contact with or in proximity to a subject; and
deriving a predetermined parameter indicating a body state of the subject based on the acquired sensor information.
